(19) United States
(12) Patent Application Publication
(10) Pub. No.: US 2025/0312176 A1
(43) Pub. Date: Oct. 9, 2025

(54) APPARATUS AND METHODS FOR LOADING A REPLACEMENT HEART VALVE IMPLANT

(71) Applicant: Boston Scientific Scimed, Inc., Maple Grove, MN (US)

(72) Inventors: John Lardner, Galway (IE); Tim O'Connor, Galway (IE); Pearse A. Coffey, Galway (IE)

(73) Assignee: BOSTON SCIENTIFIC SCIMED, INC., MAPLE GROVE, MN (US)

(21) Appl. No.: 19/098,251

(22) Filed: Apr. 2, 2025

Related U.S. Application Data
(60) Provisional application No. 63/573,904, filed on Apr. 3, 2024.

(51) Int. Cl. A61F 2/95; A61F 2/24

(52) U.S. Cl. CPC A61F 2/9524; A61F 2/2436

(57) ABSTRACT

An apparatus for loading a heart valve implant in an implant delivery system includes a stent crimping device including a central aperture and at least one radial compression mechanism, and a loading tube including a support structure attached to the loading tube and couplable to the crimping device. A method of loading the implant in the delivery system includes positioning the implant over the delivery system within the central aperture, compressing a distal portion of the implant, translating a distal sheath of the delivery system over the distal portion, translating the crimping device off the delivery system, sliding the loading tube over a proximal portion of the implant, translating the crimping device over the distal sheath and coupling the support structure to the crimping device, positioning a proximal sheath of the delivery system adjacent the crimping device, compressing the proximal portion, and translating the proximal sheath over the proximal portion.

### CROSS-REFERENCE TO RELATED APPLICATION

This application is a continuation of U.S. Patent Application Ser. No. 63/573,904, filed Apr. 3, 2024, entitled “APPARATUS AND METHODS FOR LOADING A REPLACEMENT HEART VALVE IMPLANT”, which is incorporated by reference herein in its entirety.

### TECHNICAL FIELD

The disclosure relates generally to medical devices and more particularly to medical devices that are adapted for implanting stents and medical devices including a stent component.

### BACKGROUND

A wide variety of intracorporeal medical devices and/or implants have been developed for medical use including artificial heart valve implants for repair or replacement of diseased heart valves. Loading heart valve implants into a delivery system can be difficult and time consuming. Of the known medical devices, systems, and methods, each has certain advantages and disadvantages. There is an ongoing need to provide alternative devices, systems, and methods for loading medical devices and/or heart valve implants into a delivery system.

### SUMMARY

In one example, an apparatus for loading a replacement heart valve implant in an implant delivery system may comprise a stent crimping device configured to radially compress a portion of the replacement heart valve implant, the stent crimping device comprising a central aperture and at least one radial compression mechanism, and a loading tube comprising a support structure fixedly attached to a medial portion of the loading tube, wherein the support structure is configured to couple to the stent crimping device.

In addition, or alternatively, to any example described herein, coupling the support structure to the stent crimping device aligns a lumen of the loading tube with the central aperture of the stent crimping device.

In addition, or alternatively, to any example described herein, the support structure comprises a plurality of legs extending radially outward from the loading tube. In addition, or alternatively, to any example described herein, each leg of the plurality of legs comprises an axial portion extending parallel to the lumen of the loading tube.

In addition, or alternatively, to any example described herein, the axial portion is configured to releasably couple to the stent crimping device.

In addition, or alternatively, to any example described herein, a first end portion of the loading tube extends away from the plurality of legs in a first direction, and the plurality of legs is disposed about a second end portion of the loading tube extending in a second direction opposite the first direction.

In addition, or alternatively, to any example described herein, the first end portion flares radially outward in the first direction.

In addition, or alternatively, to any example described herein, the apparatus may further comprise a funnel portion releasably couplable to the stent crimping device.

In addition, or alternatively, to any example described herein, the support structure is configured to couple to a first end of the stent crimping device and the funnel portion is configured to extend from a second end of the stent crimping device opposite the first end.

In addition, or alternatively, to any example described herein, and in a second example, a method of loading a replacement heart valve implant in an implant delivery system may comprise: positioning a replacement heart valve implant over an implant delivery system disposed within a central aperture of a stent crimping device such that a proximal end of a distal sheath of the implant delivery system is disposed adjacent a first end of the stent crimping device; radially compressing a distal portion of the replacement heart valve implant with the stent crimping device; translating the proximal end of the distal sheath of the implant delivery system over the distal portion of the replacement heart valve implant; translating the stent crimping device distally off of the implant delivery system; positioning a distal end of a proximal sheath of the implant delivery system adjacent the first end of the stent crimping device; radially compressing a proximal portion of the replacement heart valve implant with the stent crimping device; and translating the distal end of the proximal sheath of the implant delivery system over the proximal portion of the replacement heart valve implant.

In addition, or alternatively, to any example described herein, positioning the distal end of the proximal sheath of the implant delivery system adjacent the first end of the stent crimping device comprises advancing the distal sheath into the first end of the stent crimping device toward a second end of the stent crimping device.

In addition, or alternatively, to any example described herein, the stent crimping device comprises at least one radial compression mechanism.

In addition, or alternatively, to any example described herein, the stent crimping device comprises a funnel portion releasably couplable to a housing of the stent crimping device such that the funnel portion extends away from the housing to an open end of the funnel portion in a direction opposite the first end of the stent crimping device.

In addition, or alternatively, to any example described herein, and in a third example, a method of loading a replacement heart valve implant in an implant delivery system may comprise: positioning a replacement heart valve implant over an implant delivery system disposed within a central aperture of a stent crimping device such that a proximal end of a distal sheath of the implant delivery system is disposed adjacent a first end of the stent crimping device; radially compressing a distal portion of the replacement heart valve implant with the stent crimping device; translating the proximal end of the distal sheath of the implant delivery system proximally over the distal portion of the replacement heart valve implant; translating the stent crimping device distally off of the implant delivery system; sliding a loading tube comprising a support structure fixedly attached to a medial portion of the loading tube proximally over the distal sheath and a proximal portion of the replacement heart valve implant; translating the stent crimping device proximally over the distal sheath and coupling the support structure to the first end of the stent crimping device; positioning a distal end of a proximal sheath of the implant delivery system adjacent the first end of the stent crimping device; radially compressing the proximal portion of the replacement heart valve implant with the stent crimping device; and translating the distal end of the proximal sheath of the implant delivery system distally over the proximal portion of the replacement heart valve implant.

In addition, or alternatively, to any example described herein, the support structure comprises a plurality of legs configured to releasably couple to the first end of the stent crimping device.

In addition, or alternatively, to any example described herein, each leg of the plurality of legs comprises an axial portion extending parallel to the lumen of the loading tube to a distal end disposed distally of the medial portion of the loading tube.

In addition, or alternatively, to any example described herein, the distal end of each leg of the plurality of legs comprises a distal hook configured to snap onto the first end of the stent crimping device.

In addition, or alternatively, to any example described herein, the loading tube comprises a first end portion extending away from the support structure in a first direction and a second end portion extending in a second direction opposite the first direction, wherein the first end portion flares radially outward in the first direction. In addition, or alternatively, to any example described herein, sliding the loading tube proximally over the distal sheath and the proximal portion of the replacement heart valve implant comprises sliding the first end portion proximally over the distal sheath and the proximal portion of the replacement heart valve implant such that a proximalmost end of the replacement heart valve implant is disposed proximal of the first end portion.

In addition, or alternatively, to any example described herein, the method may further comprise: before translating the stent crimping device proximally over the distal sheath and coupling the support structure to the first end of the stent crimping device, translating the distal end of the proximal sheath distally over the proximalmost end of the replacement heart valve implant.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of a replacement heart valve system including the replacement heart valve implant and an implant delivery system;

FIG. 3 illustrates selected aspects of an apparatus for loading the replacement heart valve implant in the implant delivery system;

FIGS. 4-7 illustrate selected aspects of a method of loading the replacement heart valve implant in the implant delivery system;

FIG. 8 illustrates selected aspects of the replacement heart valve system with the replacement heart valve implant partially loaded in the implant delivery system;

FIG. 9 illustrates a loading tube associated with the apparatus for loading the replacement heart valve implant in the implant delivery system;

FIGS. 10-15 illustrate selected aspects of the method of loading the replacement heart valve implant in the implant delivery system; and

FIG. 16 illustrates the apparatus removed from the replacement heart valve system after loading the replacement heart valve implant in the implant delivery system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s).

Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently —such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

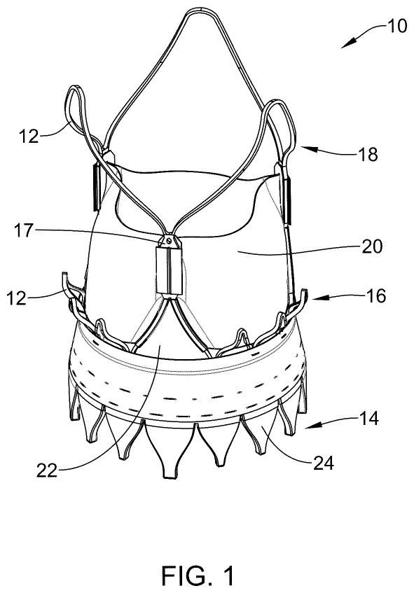

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

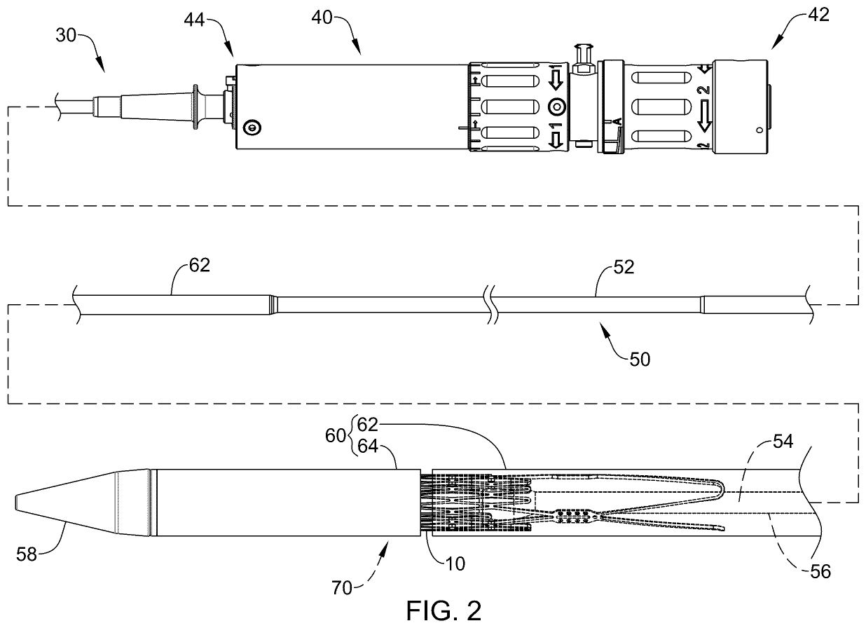

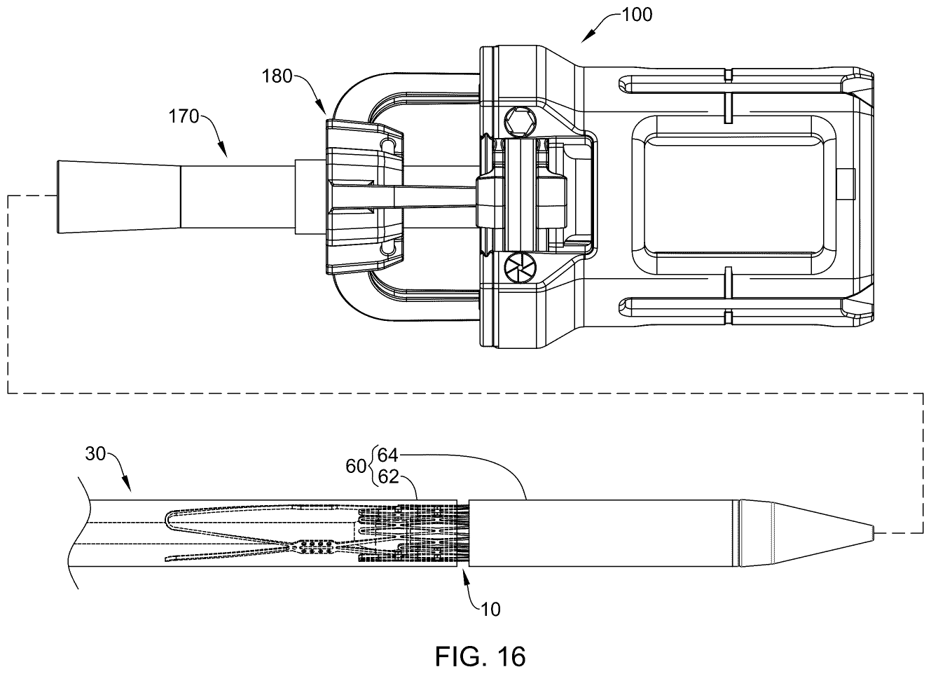

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The replacement heart valve implant 10 and/or the expandable framework 12 may be configured to shift between a radially collapsed configuration (e.g., FIGS. 2, 16) and a radially expanded configuration (e.g., FIG. 1). In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations, including combinations thereof, are also contemplated.

In some embodiments, the expandable framework 12 may define a lower crown 14 proximate and/or at the inflow end, an upper crown 16 proximate and/or at the outflow end, and a plurality of stabilization arches 18 extending downstream from the outflow end. In some embodiments, the plurality of stabilization arches 18 may extend downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the replacement heart valve implant 10 may include a proximal portion and a distal portion. In some embodiments, orientation of the replacement heart valve implant 10 may be related to an implant delivery system 30 (e.g., FIG. 2) and/or a direction of implantation relative to a target site (e.g., a native heart valve). In some embodiments, the proximal portion may include the outflow end and/or the plurality of stabilization arches 18. In some embodiments, the proximal portion may include the upper crown 16. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12 at a plurality of posts 17 to form and/or define a plurality of commissures. In addition, or alternatively, in some embodiments, the plurality of valve leaflets 20 may be coupled, secured, and/or fixedly attached to the expandable framework 12 proximate and/or at other locations, such as the inflow end, the lower crown 14, etc. The plurality of valve leaflets 20 may be configured to shift between an open position and a closed position. The plurality of valve leaflets 20 may be configured to substantially restrict fluid flow through the replacement heart valve implant 10 in the closed position. The plurality of valve leaflets 20 may move apart from each other and/or radially outward within the central lumen in the open position to permit fluid flow through the replacement heart valve implant 10 and/or the central lumen.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated. In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along an outer surface of the expandable framework 12. In some embodiments, the outer skirt 24 may be disposed at and/or adjacent the lower crown. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 23 millimeters (mm) (0.905 inches), about 25 mm (0.984 inches), about 27 mm (1.063 inches), about 30 mm (1.181 inches), etc. in an unconstrained configuration (e.g., in the radially expanded configuration). In some embodiments, the expandable framework 12 and/or the replacement heart valve implant 10 may have an outer extent of about 10 mm (0.394 inches), about 9 mm (0.354 inches), about 8 mm (0.315 inches), about 7 mm (0.276 inches), about 6 mm (0.236 inches), etc. in the radially collapsed configuration. Other configurations are also contemplated.

FIG. 2 illustrates selected aspects of a replacement heart valve system comprising the replacement heart valve implant 10 and an implant delivery system 30 for delivering the replacement heart valve implant 10 to a native heart valve. It should be noted that FIG. 2 includes at least one change of scale (e.g., all parts of the figure are not drawn to the same scale) to improve viewability and show additional detail of selected aspects of the implant delivery system 30. Additionally, the expandable framework 12 is shown in FIG. 2 in the radially collapsed configuration and some elements of the replacement heart valve implant 10 are omitted to improve clarity.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate about a central longitudinal axis of the implant delivery system 30 and/or the handle 40. Other configurations are also contemplated.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration. The elongate shaft assembly 50 may include an outer tubular member 52 extending distally from the handle 40 and an inner shaft 54 extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob and/or the second rotatable knob. In some embodiments, the handle 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in a closed configuration of the implant holding portion 60. In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 in the radially collapsed configuration when the implant holding portion 60 is in the closed configuration. In some embodiments, the replacement heart valve implant 10 may be releasably coupled to and/or releasably engaged with the intermediate tubular member 56 and/or a stent holder 70 when the replacement heart valve implant 10 is constrained within the implant holding portion 60 of the implant delivery system 30 in the radially collapsed configuration.

In some embodiments, the proximal sheath 62 may be configured to cover the proximal portion of the replacement heart valve implant 10 in the radially collapsed configuration when the implant holding portion 60 is in the closed configuration, and the distal sheath 64 may be configured to cover the distal portion of the replacement heart valve implant 10 in the radially collapsed configuration when the implant holding portion 60 is in the closed configuration. In some embodiments, the replacement heart valve implant 10 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10. In some embodiments, the proximal sheath 62 may be axially spaced apart from the distal sheath 64 in the closed configuration by less than 15% of an overall length of the replacement heart valve implant 10. In some embodiments, the proximal sheath 62 may be axially spaced apart from the distal sheath 64 in the closed configuration by less than 10% of an overall length of the replacement heart valve implant 10. In some embodiments, the proximal sheath 62 may be axially spaced apart from the distal sheath 64 in the closed configuration by less than 5% of an overall length of the replacement heart valve implant 10. Other configurations are also contemplated.

In some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the implant delivery system 30. In some embodiments, the stent holder 70 may include at least one projection configured to engage the expandable framework 12 in the radially collapsed configuration.

In use, the implant delivery system 30 may be advanced percutaneously through the vasculature to a position adjacent to the treatment site (e.g., the native valve annulus). For example, the implant delivery system 30 may be advanced through the vasculature and across the aortic arch to a position adjacent to the aortic valve. Alternative approaches to treat a defective aortic valve and/or other heart valve(s) are also contemplated with the implant delivery system 30.

A desired insertion depth may be selected to maximize radially outward force of the expandable framework 12 within the native heart valve (e.g., the aortic valve). Positioning the replacement heart valve implant 10 at the desired insertion depth and/or within a maximum tolerance from the desired insertion depth, the replacement heart valve implant 10 and/or the expandable framework 12 may exhibit optimal arching within the native heart valve (e.g., the aortic valve) and thereby prevent migration of the replacement heart valve implant 10 and/or the expandable framework 12 downstream (or upstream).

Positioning the replacement heart valve implant 10 and/or the expandable framework 12 within the native heart valve (e.g., the aortic valve) may be accomplished by locating a primary visual indicator (e.g., a marker band, etc.) relative to the native heart valve (e.g., the aortic valve). During visualization, the native heart valve (e.g., the aortic valve) may be identified and/or visualized under fluoroscopy using known means and/or methods, such as contrast injection.

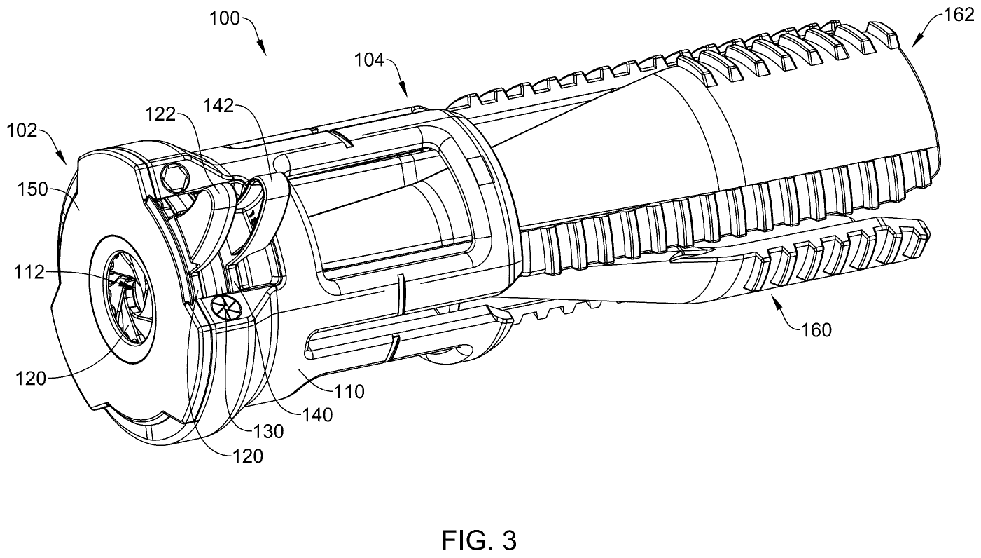

FIGS. 3-7 illustrate selected aspects of an apparatus and methods for loading the replacement heart valve implant 10 (e.g., FIG. 1) in the implant delivery system 30 (e.g., FIG. 2). It should be noted that in any given figure, some elements or features of the apparatus are not shown, are not shown in their entirety, or are shown schematically to improve clarity. Additionally, each reference number identified on the figures may not be expressly discussed with respect to that figure but may be discussed elsewhere in the disclosure and may be shown to improve clarity and/or to show relative positioning of selected elements and/or features.

The apparatus may comprise a stent crimping device 100 configured to radially compress a portion of the replacement heart valve implant 10. The stent crimping device 100 may comprise a housing 110. The stent crimping device 100 and/or the housing 110 may comprise a first end 102 and a second end 104 opposite the first end 102. The stent crimping device 100 may comprise a central aperture 112 aligned with and/or disposed coaxially with a central longitudinal axis of the stent crimping device 100 and/or the housing 110.

In some embodiments, the apparatus may comprise a funnel portion 160 releasably couplable to the stent crimping device 100. In some embodiments, the funnel portion 160 may be releasably couplable to the housing 110 of the stent crimping device 100 such that the funnel portion 160 extends away from the housing 110 to an open end 162 of the funnel portion 160 in a direction opposite the first end 102 of the stent crimping device 100. In some embodiments, the funnel portion 160 may be configured to extend from the second end 104 of the stent crimping device 100 opposite the first end 102 of the stent crimping device 100.

The stent crimping device 100 may comprise at least one radial compression mechanism. In some embodiments, the at least one radial compression mechanism may comprise a first radial compression mechanism 120 positioned adjacent and/or at least partially within the housing 110. In some embodiments, the at least one radial compression mechanism may comprise a second radial compression mechanism 140 positioned adjacent and/or at least partially within the housing 110.

In some embodiments, the stent crimping device 100 may comprise a spacer plate 130 positioned adjacent and/or at least partially within the housing 110. In some embodiments, the spacer plate 130 may be disposed between the first radial compression mechanism 120 and the second radial compression mechanism 140. The spacer plate 130 may comprise a central opening 132 formed therein. In some embodiments, the central opening 132 of the spacer plate 130 may be aligned with and/or may be positioned coaxially relative to the central aperture 112 and/or the central longitudinal axis of the stent crimping device 100 and/or the housing 110. In at least some embodiments, the spacer plate 130 may be nonrotatable relative to the housing 110. In some embodiments, the spacer plate 130 may comprise one or more projections (not shown) configured to engage with the first radial compression mechanism 120 and/or the second radial compression mechanism 140. In some alternative embodiments, the stent crimping device 100 may be devoid of the spacer plate 130.

In some embodiments, the stent crimping device 100 may comprise a cover plate 150 positioned adjacent the housing 110. In some embodiments, the cover plate 150 may be positioned at least partially within the housing 110. In some embodiments, the cover plate 150 may comprise a cover plate opening 152 aligned with and/or may be positioned coaxially relative to the central aperture 112 and/or the central longitudinal axis of the stent crimping device 100 and/or the housing 110. In some embodiments, the cover plate 150 may be disposed proximate and/or at the first end 102 of the stent crimping device 100. In some embodiments, the cover plate 150 may be removably secured to the housing 110. In some embodiments, the cover plate 150 may be non-rotatable relative to the housing 110. In one example, the cover plate 150 may be removably secured to the housing 110 using one or more fasteners (not shown). In another example, the cover plate 150 and/or one or more protrusions extending from the cover plate 150 may be configured to engage one or more slots or other features formed in the housing 110 to removably secure the cover plate 150 to the housing 110. Other configurations are also contemplated.

In some embodiments, the first radial compression mechanism 120 may comprise and/or may be formed as a first iris comprising a first plurality of arms. The first radial compression mechanism 120 may comprise a first lever 122 extending radially outward from the housing 110. The first lever 122 may be configured to move relative to the housing 110 to produce relative movement of the first radial compression mechanism 120 and/or elements thereof. The first lever 122 may be configured to move circumferentially relative to the housing 110 to produce relative movement of the first radial compression mechanism 120 and/or elements thereof. In some embodiments, relative movement of the first radial compression mechanism 120 and/or between the first radial compression mechanism 120 and/or elements thereof and the housing 110 may cause and/or produce a radial reduction in a first central opening 126 of the first radial compression mechanism 120. In some embodiments, relative movement of the first plurality of arms may cause the first iris to change size.

In some embodiments, the second radial compression mechanism 140 may comprise and/or may be formed as a second iris comprising a second plurality of arms. The second radial compression mechanism 140 may comprise a second lever 142 extending radially outward from the housing 110. The second lever 142 may be configured to move relative to the housing 110 to produce relative movement of the second radial compression mechanism 140 and/or elements thereof. The second lever 142 may be configured to move circumferentially relative to the housing 110 to produce relative movement of the second radial compression mechanism 140 and/or elements thereof. In some embodiments, relative movement of the second radial compression mechanism 140 and/or between the second radial compression mechanism 140 and/or elements thereof and the housing 110 may cause and/or produce a radial reduction in a second central opening 146 of the second radial compression mechanism 140. In some embodiments, relative movement of the second plurality of arms may cause the second iris to change size.

In some embodiments, the first radial compression mechanism 120 may be movable with respect to the housing 110, the spacer plate 130, and/or the cover plate 150. In some embodiments, at least a portion of the first radial compression mechanism 120 may be configured to rotate relative to the housing 110, the spacer plate 130, and/or the cover plate 150. In some embodiments, the second radial compression mechanism 140 may be movable with respect to the housing 110, the spacer plate 130, and/or the cover plate 150. In some embodiments, at least a portion of the second radial compression mechanism 140 may be configured to rotate relative to the housing 110, the spacer plate 130, and/or the cover plate 150. In some embodiments, at least a portion of the second radial compression mechanism 140 may be configured to rotate relative to the first radial compression mechanism 120. Accordingly, in at least some embodiments, the first radial compression mechanism 120 and the second radial compression mechanism 140 may be movable independently of each other. In some embodiments, the first radial compression mechanism 120 may be movable and/or may be configured to rotate relative to the housing 110, the spacer plate 130, and/or the cover plate 150 independently of the second radial compression mechanism 140. In some embodiments, the second radial compression mechanism 140 may be movable and/or may be configured to rotate relative to the housing 110, the spacer plate 130, and/or the cover plate 150 independently of the first radial compression mechanism 120.

In some embodiments, the first radial compression mechanism 120 and the second radial compression mechanism 140 may be movable and/or may be configured to rotate relative to the housing 110, the spacer plate 130, and/or the cover plate 150 together, in tandem, and/or simultaneously. In some embodiments, the first radial compression mechanism 120 and the second radial compression mechanism 140 may be movable and/or may be configured to rotate relative to the housing 110, the spacer plate 130, and/or the cover plate 150 together, in tandem, and/or simultaneously at one time, in one circumferential direction, or during a particular step of a method disclosed herein, and the first radial compression mechanism 120 and the second radial compression mechanism 140 may be movable and/or may be configured to rotate relative to the housing 110, the spacer plate 130, and/or the cover plate 150 independently of each other at another time, in another direction, or during another particular step of a method disclosed herein. Other configurations are also contemplated.

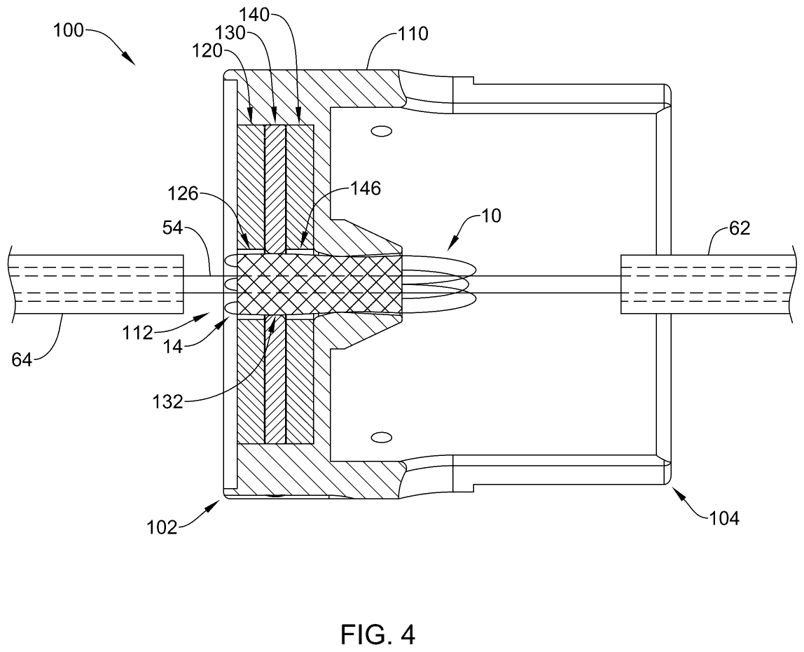

A method of loading the replacement heart valve implant 10 in the implant delivery system 30 may comprise positioning the replacement heart valve implant 10 over the implant delivery system 30 and/or selected components thereof within the central aperture 112 of the stent crimping device 100 such that a proximal end of the distal sheath 64 of the implant delivery system 30 is disposed adjacent the first end 102 of the stent crimping device 100 and/or the housing 110, as seen in FIG. 4. In FIGS. 4-7, the funnel portion 160 is not shown, but may be present. For example, the funnel portion 160 may be used to translate the distal portion of the replacement heart valve implant 10 into the central aperture 112 of the stent crimping device 100, the first central opening 126 of the first radial compression mechanism 120, the central opening 132 of the spacer plate 130, and/or the second central opening 146 of the second radial compression mechanism 140, while shifting at least a portion of the replacement heart valve implant 10 from the radially expanded configuration (e.g., FIG. 1) toward the radially collapsed configuration.

In some embodiments, the implant delivery system 30 may be inserted into the central aperture 112 before the distal portion of the replacement heart valve implant 10 is translated into the central aperture 112 of the stent crimping device 100, the first central opening 126 of the first radial compression mechanism 120, the central opening 132 of the spacer plate 130, and/or the second central opening 146 of the second radial compression mechanism 140. In some embodiments, the distal portion of the replacement heart valve implant 10 may be translated into the central aperture 112 of the stent crimping device 100, the first central opening 126 of the first radial compression mechanism 120, the central opening 136 of the spacer plate 130, and/or the second central opening 146 of the second radial compression mechanism 140 before the implant delivery system 30 is inserted through the central aperture 112 and/or the replacement heart valve implant 10. In some embodiments, the implant delivery system 30 and the distal portion of the replacement heart valve implant 10 may be translated into the central aperture 112 of the stent crimping device 100, the first central opening 126 of the first radial compression mechanism 120, the central opening 136 of the spacer plate 130, and/or the second central opening 146 of the second radial compression mechanism 140 simultaneously.

In at least some embodiments, translating the distal portion of the replacement heart valve implant 10 into the central aperture 112 of the stent crimping device 100, the first central opening 126 of the first radial compression mechanism 120, the central opening 136 of the spacer plate 130, and/or the second central opening 146 of the second radial compression mechanism 140 may partially radially compress the distal portion of the replacement heart valve implant 10. While not expressly illustrated, partially radially compressing the distal portion of the replacement heart valve implant 10 with the stent crimping device 100 may comprise radially compressing the distal portion of the replacement heart valve implant 10 onto and/or into engagement with the stent holder 70 (not shown).

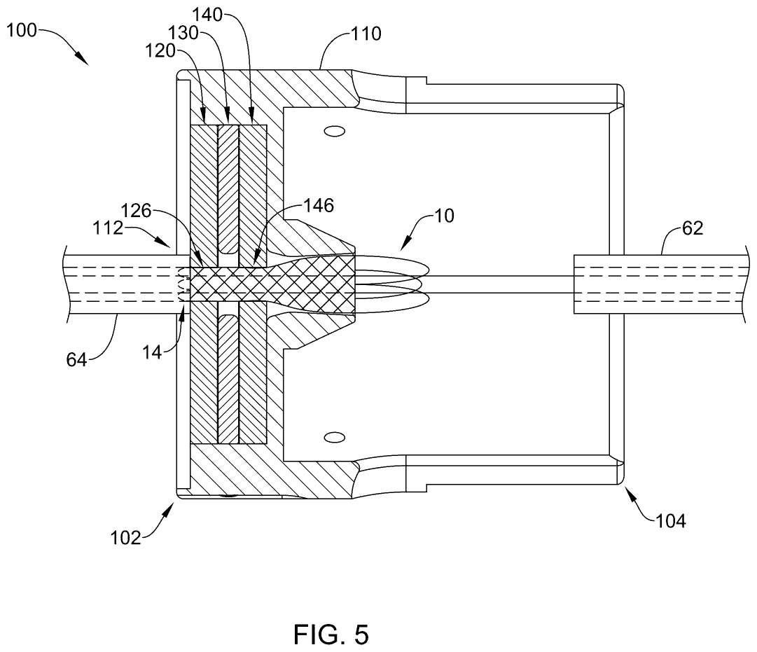

The method of loading the replacement heart valve implant 10 in the implant delivery system 30 may comprise radially compressing a portion of the replacement heart valve implant 10 toward and/or to the radially collapsed configuration with the stent crimping device 100 (e.g., with the at least one radial compression mechanism, with the first radial compression mechanism, with the second radial compression mechanism, etc.). The method of loading the replacement heart valve implant 10 in the implant delivery system 30 may comprise radially compressing the distal portion of the replacement heart valve implant 10 toward and/or to the radially collapsed configuration with the stent crimping device 100 (e.g., with the at least one radial compression mechanism, with the first radial compression mechanism, with the second radial compression mechanism, etc.), as seen in FIG. 5.

In some embodiments, radially compressing the distal portion of the replacement heart valve implant 10 toward and/or to the radially collapsed configuration may comprise actuating the at least one radial compression mechanism, the first radial compression mechanism 120, and/or the second radial compression mechanism 140 to apply a radially inward force against the distal portion of the replacement heart valve implant 10. In some embodiments, actuating the at least one radial compression mechanism, the first radial compression mechanism 120, and/or the second radial compression mechanism 140 may comprise moving the first lever 122 and/or the second lever 142 relative to the housing 110. Other configurations are also contemplated.

In some embodiments, the method of loading the replacement heart valve implant 10 in the implant delivery system 30 may comprise, after radially compressing the distal portion of the replacement heart valve implant 10, positioning and/or engaging the proximal end of the distal sheath 64 against the at least one radial compression mechanism and/or the first radial compression mechanism 120, thereby capturing a distalmost end of the replacement heart valve implant 10 and/or the lower crown 14 of the expandable framework 12 therein, as seen in FIG. 5.

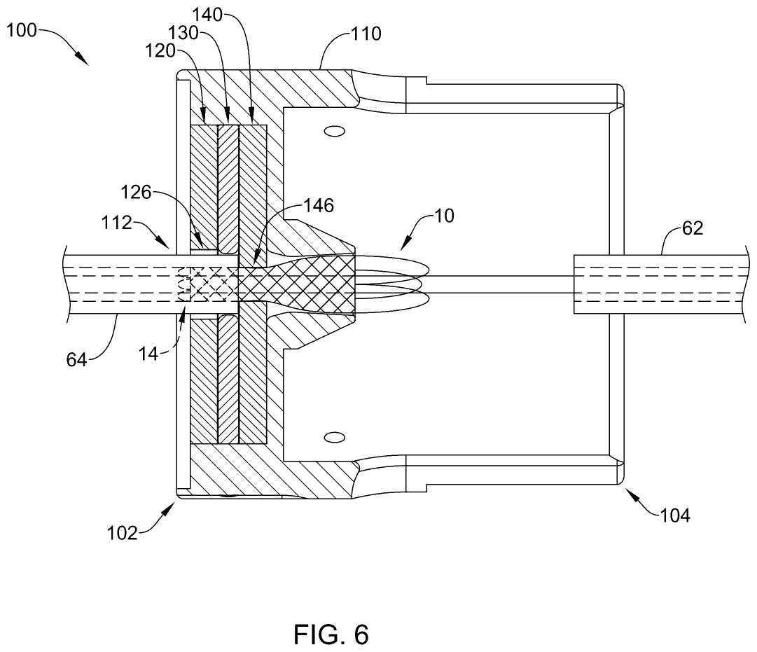

After positioning the proximal end of the distal sheath 64 against the at least one radial compression mechanism and/or the first radial compression mechanism 120, the method may comprise de-actuating the first radial compression mechanism 120 to open the first central opening 126, and thereafter, moving the proximal end of the distal sheath 64 into the at least one radial compression mechanism and/or the first radial compression mechanism 120 over the distal portion of the replacement heart valve implant 10 such that the distal portion of the replacement heart valve implant 10 disposed within the at least one radial compression mechanism and/or the first radial compression mechanism 120 is disposed within the distal sheath 64, as seen in FIG. 6. In some embodiments, the distal sheath 64 has an inner diameter less than an outer diameter of the distal portion of the replacement heart valve implant 10 when the replacement heart valve implant 10 is in the radially expanded configuration. As such, radial compression of the replacement heart valve implant 10 is required in order to move the replacement heart valve implant 10 into the distal sheath 64.

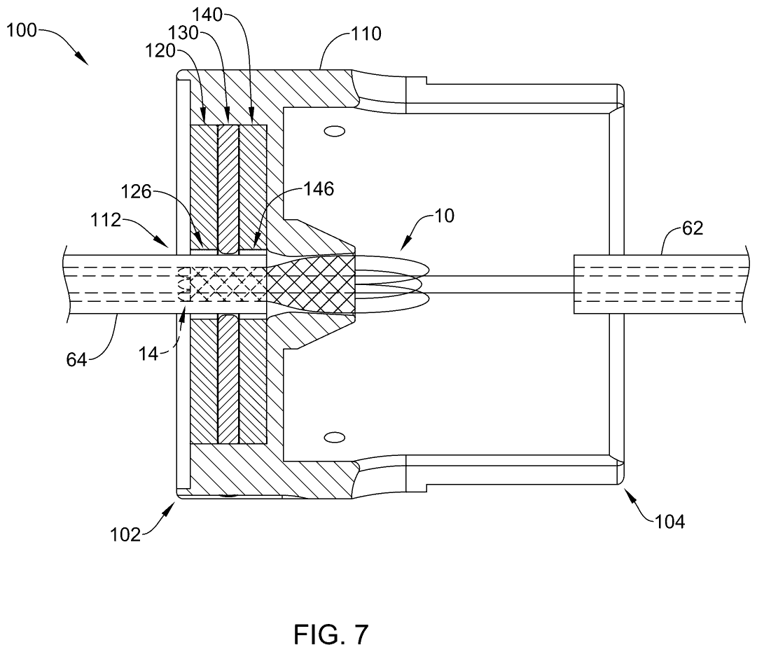

In some embodiments, after moving the distal sheath 64 into the at least one radial compression mechanism and/or the first radial compression mechanism 120 over the replacement heart valve implant 10, the method may further comprise de-actuating the at least one radial compression mechanism and/or the second radial compression mechanism 140 to open the second central opening 146, and thereafter, moving the proximal end of the distal sheath 64 into the at least one radial compression mechanism and/or the second radial compression mechanism 140 over the replacement heart valve implant 10 in the compressed configuration such that the distal portion of the replacement heart valve implant 10 disposed within the at least one radial compression mechanism and/or the second radial compression mechanism 140 is disposed within the distal sheath 64, as seen in FIG. 7.

In some embodiments, the process described herein with respect to FIGS. 4-7 may be repeated if more of the distal portion of the replacement heart valve implant 10 needs to be radially compressed and/or moved into the distal sheath 64.

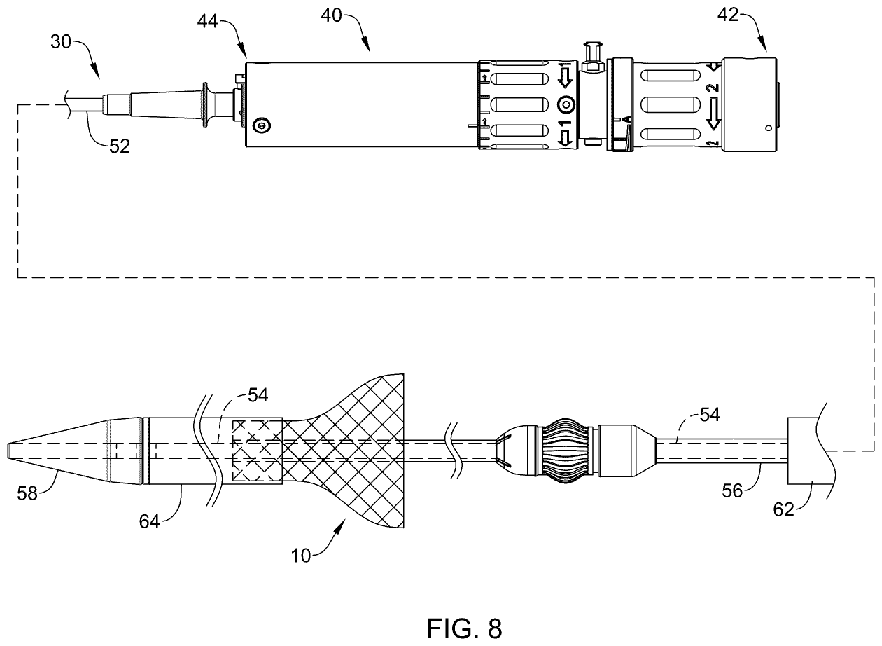

In some embodiments, the method of loading the replacement heart valve implant 10 in the implant delivery system 30 may comprise translating the stent crimping device 100 distally off of the implant delivery system 30, as seen in FIG. 8. At this point in the process and/or method of loading the replacement heart valve implant 10 in the implant delivery system 30, the distal portion of the replacement heart valve implant 10 may be disposed within the distal sheath 64 and the proximal portion of the replacement heart valve implant 10 may be disposed outside of the distal sheath 64 and/or may extend proximally from the distal sheath 64. In at least some embodiments, prior to translating the stent crimping device 100 distally off of the implant delivery system 30, the funnel portion 160 may be decoupled and/or detached from the housing 110. In some embodiments, after decoupling and/or detaching the funnel portion 160 from the housing 110, the funnel portion 160 may be slid proximally over the proximal sheath 62 and/or the outer tubular member 52. In some embodiments, the funnel portion 160 may be configured to remain on the outer tubular member 52 and/or the elongate shaft assembly 50 (e.g., FIG. 2) until the replacement heart valve implant 10 is disposed within the implant holding portion 60 (e.g., FIGS. 2, 16) in the radially collapsed configuration.

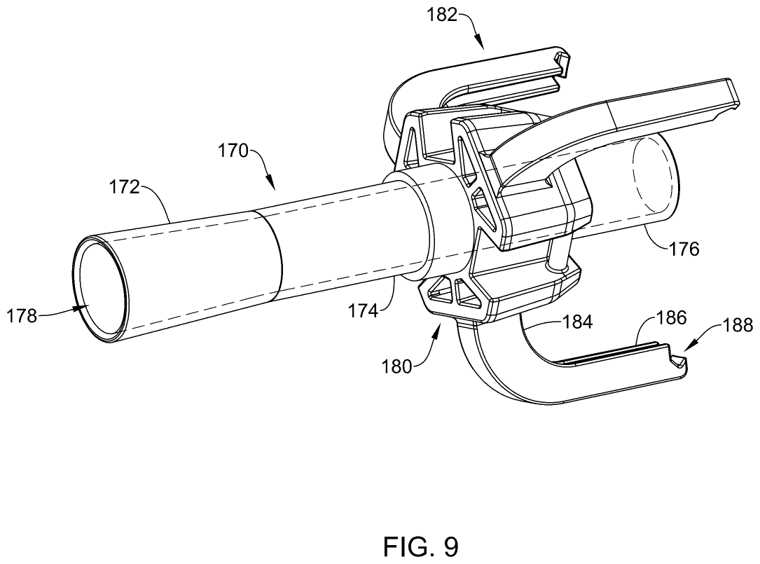

In some embodiments, the apparatus for loading the replacement heart valve implant 10 in the implant delivery system 30 may comprise a loading tube 170. FIG. 9 illustrates selected aspects of the loading tube 170. The loading tube 170 may comprise a first end portion 172, a medial portion 174, and a second end portion 176. The first end portion 172 may be spaced apart from the second end portion 176 by the medial portion 174. The loading tube 170 may comprise a lumen 178 extending axially therethrough.

In some embodiments, the loading tube 170 may comprise a support structure 180 fixedly attached to the medial portion 174 of the loading tube 170. In some embodiments, the support structure 180 may be fixedly attached to the medial portion 174 of the loading tube 170 by adhesive bonding, welding, mechanical securement means, etc. In some embodiments, the loading tube 170 may be formed from a different material than the support structure 180. In some embodiments, the loading tube 170 may be formed from the same material as the support structure 180. In some embodiments, the support structure 180 may be integrally formed with and/or monolithically formed with the loading tube 170 and/or the medial portion 174 of the loading tube 170. In some embodiments, the loading tube 170 and/or the support structure 180 may be formed by injection molding, machining, or other suitable methods.

In at least some embodiments, the loading tube 170 may be formed from a clear polymeric material to facilitate visibility of the replacement heart valve implant 10 therein. In some embodiments, the loading tube 170 and/or the support structure 180 may be formed from polycarbonate. In some embodiments, the loading tube 170 and/or the support structure 180 may be formed from the same polycarbonate material. In some embodiments, the loading tube 170 and/or the support structure 180 may be formed from different polycarbonate materials. Some other suitable but non-limiting materials are described below.

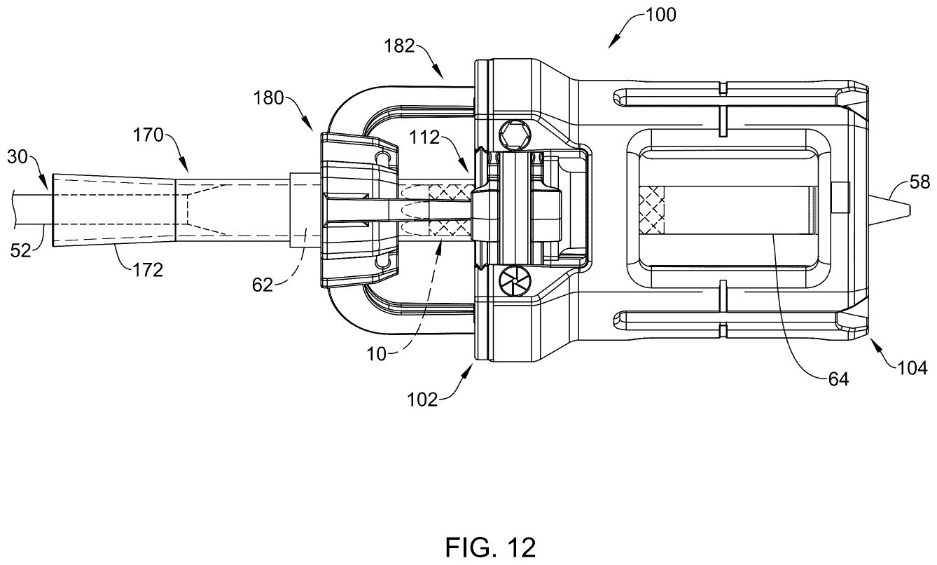

In some embodiments, the support structure 180 may be configured to couple and/or attach to the stent crimping device 100 (e.g., FIG. 12). In some embodiments, the support structure 180 may be configured to couple and/or attach to the first end 102 of the stent crimping device 100. In some embodiments, the support structure 180 may be configured to releasably couple and/or releasably attach to the stent crimping device 100. In some embodiments, the support structure 180 may be configured to releasably couple and/or releasably attach to the first end 102 of the stent crimping device 100.

In some embodiments, the support structure 180 may comprise a plurality of legs 182 extending radially outward from the loading tube 170. In some embodiments, the plurality of legs 182 may comprise three legs. In some embodiments, the plurality of legs 182 may comprise exactly three legs. In some embodiments, the plurality of legs 182 may comprise three or more legs. In some embodiments, the plurality of legs 182 may comprise four legs, five legs, or more than five legs. In some alternative embodiments, the plurality of legs may comprise two or more legs.

In some embodiments, each leg of the plurality of legs 182 may comprise a lateral portion 184 extending radially outward from the loading tube 170 and an axial portion 186 extending parallel to the loading tube 170 and/or the lumen 178 of the loading tube 170.

In some embodiments, the axial portion 186 may extend parallel to the lumen 178 of the loading tube 170 to a distal end disposed distal of the medial portion 174 of the loading tube 170. In some embodiments, the axial portion 186 may be configured to couple and/or attach to the stent crimping device 100. In some embodiments, the axial portion 186 may be configured to releasably couple and/or releasably attach to the stent crimping device 100. In some embodiments, the axial portion 186 may be configured to couple and/or attach to the first end 102 of the stent crimping device 100. In some embodiments, the axial portion 186 may be configured to releasably couple and/or releasably attach to the first end 102 of the stent crimping device 100. In at least some embodiments, the distal end of each leg of the plurality of legs 182 comprises a distal hook 188 configured to couple and/or attach to the stent crimping device 100. In some embodiments, the distal hook 188 may be configured to releasably couple and/or releasably attach to the stent crimping device 100. In some embodiments, the distal hook 188 may be configured to couple and/or attach to the first end 102 of the stent crimping device 100. In some embodiments, the distal hook 188 may be configured to releasably couple and/or releasably attach to the first end 102 of the stent crimping device 100. In some embodiments, the distal hook 188 may be configured to snap onto the housing 110 of the stent crimping device 100. In some embodiments, the distal hook 188 may be configured to snap onto the first end 102 of the stent crimping device 100. Other configurations are also contemplated.

In some embodiments, the first end portion 172 of the loading tube 170 may extend away from the support structure 180 and/or away from the plurality of legs 182 in a first direction. In some embodiments, the first end portion 172 of the loading tube 170 may flare radially outward in the first direction. In some embodiments, the second end portion 176 of the loading tube 170 may extend in a second direction opposite the first direction. In some embodiments, the plurality of legs 182 of the support structure 180 may be disposed about and/or around the second end portion 176 of the loading tube 170 extending in the second direction opposite the first direction. In some embodiments, the axial portion 186 of each leg of the plurality of legs 182 may extend from the lateral portion 184 in the second direction.

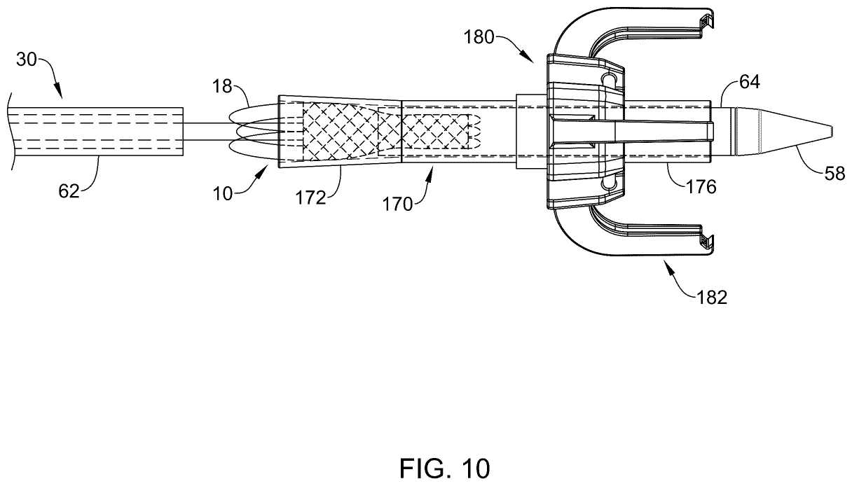

FIGS. 10-16 illustrate selected aspects of the apparatus and methods for loading the replacement heart valve implant 10 (e.g., FIGS. 1, 8) in the implant delivery system 30 (e.g., FIGS. 2, 8). It should be noted that in any given figure, some elements or features of the apparatus are not shown, are not shown in their entirety, or are shown schematically to improve clarity. Additionally, each reference number identified on the figures may not be expressly discussed with respect to that figure but may be discussed elsewhere in the disclosure and may be shown to improve clarity and/or to show relative positioning of selected elements and/or features.

In some embodiments, the method of loading the replacement heart valve implant 10 in the implant delivery system 30 may comprise sliding the loading tube 170 proximally over the distal sheath 64 of the implant delivery system 30 and the proximal portion of the replacement heart valve implant 10, as seen in FIG. 10. In some embodiments, sliding the loading tube 170 proximally over the distal sheath 64 of the implant delivery system 30 and the proximal portion of the replacement heart valve implant 10 may comprise sliding the first end portion 172 of the loading tube 170 proximally over the distal sheath 64 of the implant delivery system 30 and at least a portion of the proximal portion of the replacement heart valve implant 10 such that a proximalmost end (e.g., the plurality of stabilization arches 18 and/or a proximal portion of the plurality of stabilization arches 18) of the replacement heart valve implant 10 is disposed proximal of the first end portion 172 of the loading tube 170. In some embodiments, after sliding the loading tube 170 proximally over the distal sheath 64 and the proximal portion of the replacement heart valve implant 10, the plurality of legs 182 of the support structure 180 may extend toward the distal tip 58 and/or a distal end of the implant delivery system 30.

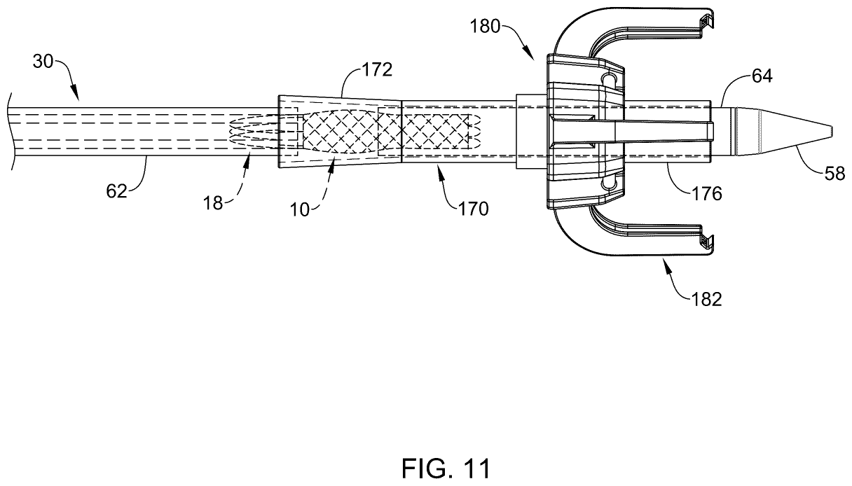

In some embodiments, the method of loading the replacement heart valve implant 10 in the implant delivery system 30 may comprise translating the distal end of the proximal sheath 62 of the implant delivery system 30 relative to and/or distally toward the loading tube 170 and/or the replacement heart valve implant 10 disposed therein. In some embodiments, the method of loading the replacement heart valve implant 10 in the implant delivery system 30 may comprise translating the distal end of the proximal sheath 62 of the implant delivery system 30 distally over the proximalmost end (e.g., the plurality of stabilization arches 18 and/or a proximal portion of the plurality of stabilization arches 18) of the replacement heart valve implant 10, as seen in FIG. 11. In some alternative embodiments, the method of loading the replacement heart valve implant 10 in the implant delivery system 30 may comprise translating the proximalmost end (e.g., the plurality of stabilization arches 18 and/or a proximal portion of the plurality of stabilization arches 18) of the replacement heart valve implant 10 proximally toward and/or into the distal end of the proximal sheath 62 of the implant delivery system 30. In some embodiments, the method of loading the replacement heart valve implant 10 in the implant delivery system 30 may comprise translating the distal end of the proximal sheath 62 of the implant delivery system 30 distally into the first end portion 172 of the loading tube 170 while translating the distal end of the proximal sheath 62 of the implant delivery system 30 distally over the proximalmost end (e.g., the plurality of stabilization arches 18 and/or a proximal portion of the plurality of stabilization arches 18) of the replacement heart valve implant 10.

In some embodiments, the method of loading the replacement heart valve implant 10 in the implant delivery system 30 may comprise translating the stent crimping device 100 proximally over the distal sheath 64 of the implant delivery system 30 and coupling and/or attaching the support structure 180 and/or the plurality of legs 182 to the stent crimping device 100, as seen in FIG. 12. In some embodiments, the method of loading the replacement heart valve implant 10 in the implant delivery system 30 may comprise translating the stent crimping device 100 proximally over the distal sheath 64 of the implant delivery system 30 and coupling and/or attaching the support structure 180 and/or the plurality of legs 182 to the first end 102 of the stent crimping device 100. In some embodiments, coupling and/or attaching the support structure 180 and/or the plurality of legs 182 to the first end 102 of the stent crimping device 100 may align the lumen 178 (e.g., FIG. 9) of the loading tube 170 with the central aperture 112 of the stent crimping device 100. In some embodiments, coupling and/or attaching the support structure 180 and/or the plurality of legs 182 to the first end 102 of the stent crimping device 100 may align the lumen 178 (e.g., FIG. 9) of the loading tube 170 coaxially with the central longitudinal axis of the stent crimping device 100 and/or the housing 110.

In some embodiments, translating the stent crimping device 100 proximally over the distal sheath 64 of the implant delivery system 30 may comprise inserting the distal tip 58 and/or the distal sheath 64 of the implant delivery system 30 into the central aperture 112 of the stent crimping device 100. In some embodiments, translating the stent crimping device 100 proximally over the distal sheath 64 of the implant delivery system 30 may comprise advancing the distal tip 58 and/or the distal sheath 64 of the implant delivery system 30 through the central aperture 112 of the stent crimping device 100. It is noted that the funnel portion 160 is not coupled to or attached to the stent crimping device 100 at this stage of the method and/or loading process. Instead, the funnel portion 160 remains disposed over the outer tubular member 52 of the implant delivery system 30 at a position proximal of the implant holding portion 60 (e.g., FIG. 16) and/or the proximal sheath 62.

In some embodiments, the loading tube 170 may be translated proximally along and/or over the proximal sheath 62 of the implant delivery system 30 as the stent crimping device 100 is translated proximally over the distal sheath 64 of the implant delivery system 30 and coupled and/or attached to the support structure 180. In some embodiments, the method of loading the replacement heart valve implant 10 in the implant delivery system 30 may comprise, before translating the stent crimping device 100 proximally over the distal sheath 64 of the implant delivery system 30 and coupling and/or attaching the support structure 180 to the first end 102 of the stent crimping device 100, translating the distal end of the proximal sheath 62 of the implant delivery system 30 distally over the proximalmost end (e.g., the plurality of stabilization arches 18 and/or a proximal portion of the plurality of stabilization arches 18) of the replacement heart valve implant 10.

In some embodiments, the method of loading the replacement heart valve implant 10 in the implant delivery system 30 may comprise positioning the distal end of the proximal sheath 62 of the implant delivery system 30 adjacent the first end 102 of the stent crimping device 100. In some embodiments, positioning the distal end of the proximal sheath 62 of the implant delivery system 30 adjacent the first end 102 of the stent crimping device 100 may comprise advancing the distal sheath 64 into the first end 102 of the stent crimping device 100 toward the second end 104 of the stent crimping device 100.

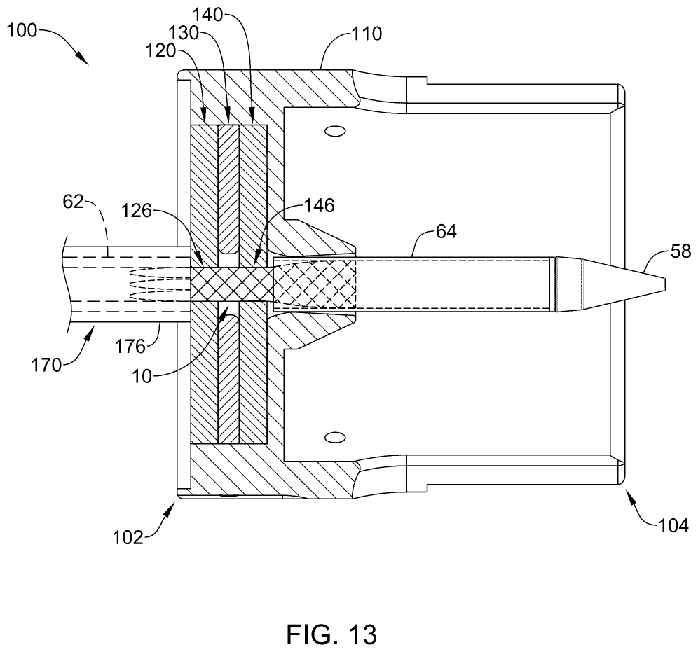

The method of loading the replacement heart valve implant 10 in the implant delivery system 30 may comprise radially compressing the proximal portion of the replacement heart valve implant 10 toward and/or to the radially collapsed configuration with the stent crimping device 100 (e.g., with the at least one radial compression mechanism, with the first radial compression mechanism, with the second radial compression mechanism, etc.), as seen in FIG. 13. In some embodiments, radially compressing the proximal portion of the replacement heart valve implant 10 toward and/or to the radially collapsed configuration may comprise actuating the at least one radial compression mechanism, the first radial compression mechanism 120, and/or the second radial compression mechanism 140 to apply a radially inward force against the proximal portion of the replacement heart valve implant 10. In some embodiments, actuating the at least one radial compression mechanism, the first radial compression mechanism 120, and/or the second radial compression mechanism 140 may comprise moving the first lever 122 and/or the second lever 142 relative to the housing 110. Other configurations are also contemplated.

In some embodiments, the method of loading the replacement heart valve implant 10 in the implant delivery system 30 may comprise, after radially compressing the proximal portion of the replacement heart valve implant 10, positioning and/or engaging the distal end of the proximal sheath 62 and/or the second end portion 176 of the loading tube 170 against the at least one radial compression mechanism and/or the first radial compression mechanism 120, thereby capturing the plurality of stabilization arches 18 and/or the upper crown 16 of the expandable framework 12 within the distal end of the proximal sheath 62, as seen in FIG. 13. It is noted that in FIGS. 13-15, the support structure 180 and/or the plurality of legs 182 is not shown.

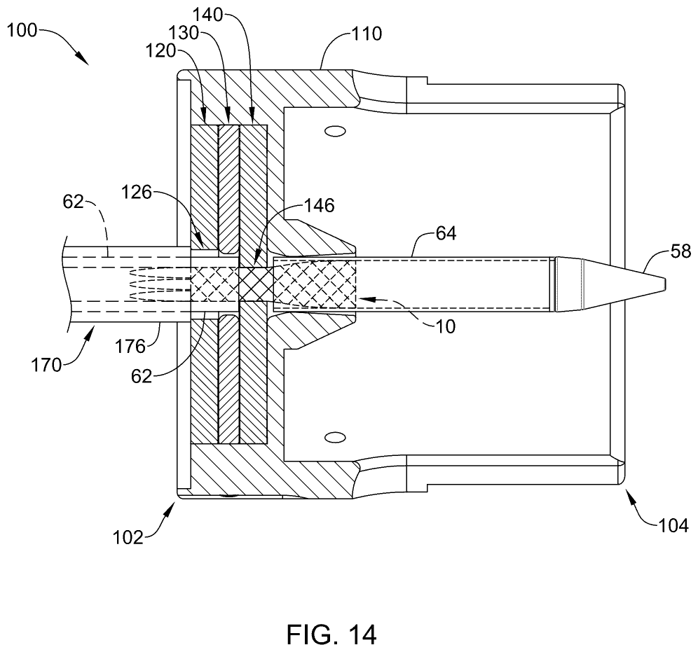

After positioning the distal end of the proximal sheath 62 and/or the second end portion 176 of the loading tube 170 against the at least one radial compression mechanism and/or the first radial compression mechanism 120, the method may comprise de-actuating the first radial compression mechanism 120 to open the first central opening 126, and thereafter, moving the distal end of the proximal sheath 62 into the at least one radial compression mechanism and/or the first radial compression mechanism 120 over the proximal portion of the replacement heart valve implant 10 such that the proximal portion of the replacement heart valve implant 10 disposed within the at least one radial compression mechanism and/or the first radial compression mechanism 120 is disposed within the proximal sheath 62, as seen in FIG. 14. In some embodiments, the proximal sheath 62 has an inner diameter less than an outer diameter of the proximal portion of the replacement heart valve implant 10 when the replacement heart valve implant 10 is in the radially expanded configuration. As such, radial compression of the replacement heart valve implant 10 is required in order to move the replacement heart valve implant 10 into the proximal sheath 62.

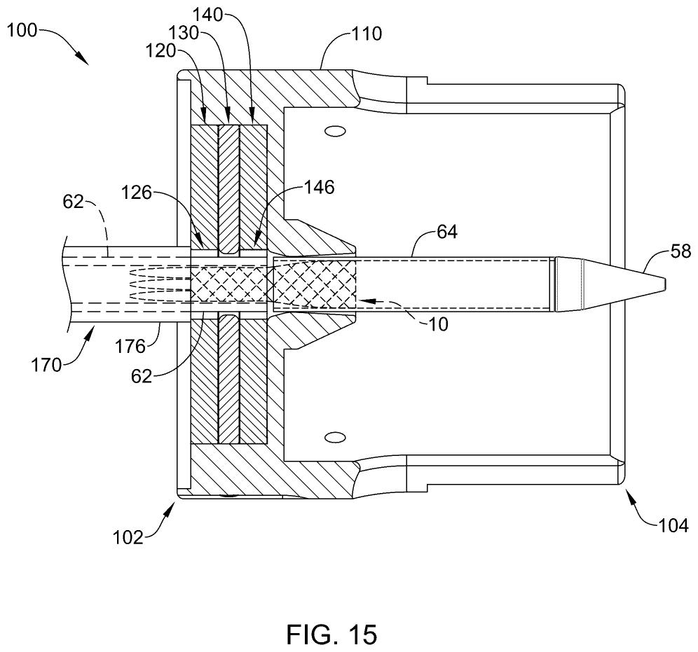

In some embodiments, after moving the proximal sheath 62 into the at least one radial compression mechanism and/or the first radial compression mechanism 120 over the replacement heart valve implant 10, the method may further comprise de-actuating the at least one radial compression mechanism and/or the second radial compression mechanism 140 to open the second central opening 146, and thereafter, moving the distal end of the proximal sheath 62 into the at least one radial compression mechanism and/or the second radial compression mechanism 140 over the replacement heart valve implant 10 in the compressed configuration such that the proximal portion of the replacement heart valve implant 10 disposed within the at least one radial compression mechanism and/or the second radial compression mechanism 140 is disposed within the proximal sheath 62, as seen in FIG. 15.

In some embodiments, the process described herein with respect to FIGS. 12-15 may be repeated if more of the proximal portion of the replacement heart valve implant 10 needs to be radially compressed and/or moved into the proximal sheath 62.

In some embodiments, the method of loading the replacement heart valve implant 10 in the implant delivery system 30 may comprise translating the apparatus, including the stent crimping device 100 and the loading tube 170, distally off of the implant delivery system 30, as seen in FIG. 16. In FIG. 16, some portions of the replacement heart valve implant 10 are omitted to improve clarity. At this point in the process and/or method of loading the replacement heart valve implant 10 in the implant delivery system 30, the distal portion of the replacement heart valve implant 10 may be disposed within the distal sheath 64 and the proximal portion of the replacement heart valve implant 10 may be disposed within the proximal sheath 62 (e.g., the replacement heart valve implant 10 may be disposed within the implant holding portion 60 in the radially collapsed configuration). Subsequently, or concurrently, the funnel portion 160 may be translated distally off of the implant delivery system 30, thereby rendering the replacement heart valve system ready for use.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the implant delivery system, the stent crimping device, the loading tube, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly (alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®,other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOXR, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An apparatus for loading a replacement heart valve implant in an implant delivery system, comprising:
a stent crimping device configured to radially compress a portion of the replacement heart valve implant, the stent crimping device comprising a central aperture and at least one radial compression mechanism; and
a loading tube comprising a support structure fixedly attached to a medial portion of the loading tube, wherein the support structure is configured to couple to the stent crimping device.

2. The apparatus of claim 1, wherein coupling the support structure to the stent crimping device aligns a lumen of the loading tube with the central aperture of the stent crimping device.

3. The apparatus of claim 1, wherein the support structure comprises a plurality of legs extending radially outward from the loading tube.

4. The apparatus of claim 3, wherein each leg of the plurality of legs comprises an axial portion extending parallel to the lumen of the loading tube.

5. The apparatus of claim 4, wherein the axial portion is configured to releasably couple to the stent crimping device.

6. The apparatus of claim 3, wherein a first end portion of the loading tube extends away from the plurality of legs in a first direction, and the plurality of legs is disposed about a second end portion of the loading tube extending in a second direction opposite the first direction.

7. The apparatus of claim 6, wherein the first end portion flares radially outward in the first direction.

8. The apparatus of claim 1, further comprising a funnel portion releasably couplable to the stent crimping device.

9. The apparatus of claim 8, wherein the support structure is configured to couple to a first end of the stent crimping device and the funnel portion is configured to extend from a second end of the stent crimping device opposite the first end.

10. A method of loading a replacement heart valve implant in an implant delivery system, comprising:
positioning a replacement heart valve implant over an implant delivery system disposed within a central aperture of a stent crimping device such that a proximal end of a distal sheath of the implant delivery system is disposed adjacent a first end of the stent crimping device;
radially compressing a distal portion of the replacement heart valve implant with the stent crimping device;
translating the proximal end of the distal sheath of the implant delivery system over the distal portion of the replacement heart valve implant;
translating the stent crimping device distally off of the implant delivery system;
positioning a distal end of a proximal sheath of the implant delivery system adjacent the first end of the stent crimping device;
radially compressing a proximal portion of the replacement heart valve implant with the stent crimping device; and
translating the distal end of the proximal sheath of the implant delivery system over the proximal portion of the replacement heart valve implant.

11. The method of claim 10, wherein positioning the distal end of the proximal sheath of the implant delivery system adjacent the first end of the stent crimping device comprises advancing the distal sheath into the first end of the stent crimping device toward a second end of the stent crimping device.

12. The method of claim 10, wherein the stent crimping device comprises at least one radial compression mechanism.

13. The method of claim 10, wherein the stent crimping device comprises a funnel portion releasably couplable to a housing of the stent crimping device such that the funnel portion extends away from the housing to an open end of the funnel portion in a direction opposite the first end of the stent crimping device.

14. A method of loading a replacement heart valve implant in an implant delivery system, comprising:
positioning a replacement heart valve implant over an implant delivery system disposed within a central aperture of a stent crimping device such that a proximal end of a distal sheath of the implant delivery system is disposed adjacent a first end of the stent crimping device;
radially compressing a distal portion of the replacement heart valve implant with the stent crimping device;
translating the proximal end of the distal sheath of the implant delivery system proximally over the distal portion of the replacement heart valve implant;
translating the stent crimping device distally off of the implant delivery system;
sliding a loading tube comprising a support structure fixedly attached to a medial portion of the loading tube proximally over the distal sheath and a proximal portion of the replacement heart valve implant;
translating the stent crimping device proximally over the distal sheath and coupling the support structure to the first end of the stent crimping device;
positioning a distal end of a proximal sheath of the implant delivery system adjacent the first end of the stent crimping device;
radially compressing the proximal portion of the replacement heart valve implant with the stent crimping device; and
translating the distal end of the proximal sheath of the implant delivery system distally over the proximal portion of the replacement heart valve implant.

15. The method of claim 14, wherein the support structure comprises a plurality of legs configured to releasably couple to the first end of the stent crimping device.

16. The method of claim 15, wherein each leg of the plurality of legs comprises an axial portion extending parallel to the lumen of the loading tube to a distal end disposed distally of the medial portion of the loading tube.

17. The method of claim 15, wherein the distal end of each leg of the plurality of legs comprises a distal hook configured to snap onto the first end of the stent crimping device.

18. The method of claim 14, wherein the loading tube comprises a first end portion extending away from the support structure in a first direction and a second end portion extending in a second direction opposite the first direction, wherein the first end portion flares radially outward in the first direction.

19. The method of claim 18, wherein sliding the loading tube proximally over the distal sheath and the proximal portion of the replacement heart valve implant comprises sliding the first end portion proximally over the distal sheath and the proximal portion of the replacement heart valve implant such that a proximalmost end of the replacement heart valve implant is disposed proximal of the first end portion.

20. The method of claim 19, further comprising:
before translating the stent crimping device proximally over the distal sheath and coupling the support structure to the first end of the stent crimping device, translating the distal end of the proximal sheath distally over the proximalmost end of the replacement heart valve implant.
